(19) United States
(12) Patent Application Publication
(10) Pub. No.: US 2008/0214814 A1
(43) Pub. Date: Sep. 4, 2008

(54) Stable ionic liquid complexes and methods for determining stability thereof

(76) Inventors: Zaiwei Li, Moreno Valley, CA (US); Yongchun Tang, Walnut, CA (US); Jihong Cheng, Arcadia, CA (US)

Correspondence Address: LADAS & PARRY, 5670 WILSHIRE BOULEVARD, SUITE 2100, LOS ANGELES, CA, 90036-5679, US

(21) Appl. No.: 11/880,106

(22) Filed: Jul. 18, 2007

Related U.S. Application Data
(60) Provisional application No. 60/831,646, filed on Jul. 18, 2006.
(60) Provisional application No. 60/832,498, filed on Jul. 21, 2006.

(51) Int. Cl. C07D 231/10; C07D 241/10; C07D 255/02

(52) U.S. Cl. 544/336; 548/373.1; 548/100

(57) ABSTRACT

Stable ionic liquid complexes capable of maintaining stability at high temperatures, under acidic and highly oxidative conditions in the presence of a transition metal, and methods for determining the stability of stable ionic liquid complexes are provided. In accordance with the disclosure herein, the stable ionic liquid complexes are derived from pyrazole, pyrazine and 1,2,4-triazole.

### CROSS REFERENCE TO RELATED APPLICATIONS

This application claims priority to U.S. Provisional Ser. No. 60/831,646 for “Stable Ionic Liquids as Media Using at High Temperature, Highly Oxidative and Strong Acidic Conditions” filed on Jul. 18, 2006 and U.S. Provisional Ser. No. 60/832,498 for “Stable Ionic Liquids as Media Using at High Temperature, Highly Oxidative and Strong Acidic Conditions” filed on Jul. 21, 2006, both of which are incorporated herein by reference in their entirety.

### STATEMENT OF GOVERNMENT SUPPORT

The present invention was made with support from the United States Government under Grant number DE-FC36-04G014276/T-103267 awarded by the U.S. Department of Energy (DOE). The United States Government has certain rights in the invention.

### BACKGROUND

1. Field

The present disclosure relates to compositions and methods involving ionic liquids which are stable under extreme conditions such as high temperature, high pH, high oxidation and in the presence of a powerful transition metal, for use in various chemical reactions and industrial processes.

2. Description of Related Art

Many chemical reactions require harsh conditions, such as high temperature, strongly acidic or oxidative environments. It has been an ongoing challenge to develop basic chemistry for selective, low-temperature, direct, oxidative conversion of alkane C—H bonds to useful functional groups. The present chemistry for a C—H conversion reaction for the homogenous catalysis of direct partial oxidation of methane to liquid products such as methanol or acetic acid requires harsh conditions. Specifically, this reaction requires high activation energy to split the strong covalent C—H bond (−105 kcal/mol). Typically, the most effective catalytic systems for direct methane oxidation require strong acid media (96% sulfuric acid (H₂SO₄) to 102% oleum or concentrated selenic acid H₂SeO₄) a high temperature (180° to 220° C.) and a powerful transition metal catalyst such as Hg(II)(1), Pt(II)(2), Au(III)(3) or Pd(II)(4) However, these harsh conditions require volumes of volatile, acidic liquids which pose a problem for the technician in the final extraction step, not to mention the safety issues for the technician, and the remaining liquid waste for the environment. (1: Periana R. et al., Science, 1993, 259, 340-343; 2: Periana R. et al., Science, 1998, 280, 560-564; 3: Jones C. J. et al., Chem. Int. Ed, 2004, 43, 4626; 4: Periana, R. et al., Science, 2003, 301, 814-817).

Ionic liquids (ILs) are charged organic salts that generally are liquids at room temperature, and are capable of dissolving many types of compounds that are relatively insoluble in aqueous or organic solvent systems. Ionic liquids are good solvents for a wide range of both inorganic and organic materials, and unusual combinations of reagents can be brought into the same phase (Welton, T. Chem. Rev, 1999, 99, 2071-2083).

Ionic liquids offer numerous advantages over conventional organic solvents for carrying out organic reactions, including very low vapor pressure, lack of flammability, and the capacity to be functionalized to suit particular reactions. Unlike conventional molten salts (for example, molten sodium chloride), most ionic liquids melt below 300 degrees Celsius. Since the melting points are low, ionic liquids can act as solvents in which reactions can be performed, and because the liquid is made of ions rather than molecules, such reactions often provide distinct selectivities and reactivities as compared to conventional organic solvents. In addition, their non-volatility results in low impact on the environment and human health, and they are recognized as solvents for “green” chemistry (Green Chemistry, 2002 (entire issue)).

U.S. patent application Ser. No. 11/228,788 to Li et al discloses the Use of Ionic Liquids as Coordination Ligands for Organometallic Catalysts. This application, which is incorporated herein by reference in its entirety, provides for ionic liquids with dissolved metal compounds as catalysts for various chemical reactions such as methane to methanol as described above.

Nonetheless, under extreme conditions, the stability of ionic liquids is a continual challenge. For example, commonly used imidazole-based ionic liquids (e.g 1-methyl-3-butyl-imidazolium chloride) decompose slowly as methane oxidation proceeds. Ring opening and deep oxidation to NH₄⁺ and CO₂ has been observed under harsh conditions (U.S. patent application Ser. No. 11/228,788), thereby eliminating the catalyzing capabilities of the ionic liquid. Or, in the least producing unwanted reaction products that are both time-consuming and often costly to remove from the desired reaction product (e.g. methanol). Therefore, ionic liquids which are stable for some reactions, are not stable in reactions requiring extreme conditions. High temperature is not the only limiting factor. Some known ionic liquids can withstand extremely high temperatures, but the combination of concentrated acid and a transition metal catalyst often makes the ionic liquid unstable at 200° C.

The present disclosure provides for ionic liquids which are stable in strongly acidic and oxidative conditions as well as at high temperatures in the presence of a powerful transition metal catalyst.

### SUMMARY

In a first embodiment of the present disclosure, a stable ionic liquid complex is provided comprising a cation and an anion; wherein the cation is one selected from the group consisting of pyrazolium, pyrazinium and 1,2,4,-triazolium and derivatives of each said cation, and the anion is derived from an acid.

In a second embodiment of the present disclosure, a method for determining the stability of an ionic liquid complex is provided, the method comprising: reacting a cation selected from the group consisting of pyrazole, pyrazine, and 1,2,4-triazole and derivatives of each said cation, with an anion derived from an acid; obtaining a reaction product containing the ionic liquid complex, and analyzing the reaction product for species other than the ionic liquid complex.

Further embodiments are disclosed throughout the specification, drawings and claims of the present disclosure.

### BRIEF DESCRIPTION OF THE DRAWINGS

FIG. 1A shows the reaction of sulfuric acid with pyrazole to produce pyrazolium bisulfate. FIG. 1B shows the reaction of sulfuric acid with pyrazine to produce pyrazinium bisulfate. FIG. 1C shows the reaction of sulfuric acid with 1,2,4-triazole to produce 1,2,4-triazolium bisulfate.

FIG. 2 shows the ¹H-NMR data of 1-methylimidazolium bisulfate after incubation at 200 degrees Celsius for 2.5 hours in the presence of PtCl₂ catalyst.

FIG. 3 shows the ¹H-NMR data of 1-methylpyrazolium bisulfate after incubation at 200 degrees Celsius for 2.5 hours in the presence of PtCl₂ catalyst.

FIG. 4 shows the ¹H-NMR data of 1-pyrazinium bisulfate after incubation at 200 degrees Celsius for 5.0 hours in the presence of PtCl₂ catalyst.

FIG. 5 shows the ¹H-NMR data of 1,2,4-triazolium bisulfate after incubation at 200 degrees Celsius for 2.5 hours in the presence of PtCl₂ catalyst.

FIG. 6 shows methanol yield as a function of H₂SO₄ concentration at 200 degrees Celsius for 2.5 hours in reactions with the Pyrianan catalyst (bpym)PtCl₂ (black diamonds) and 1-methylpyrazinium bisulfate+PtCl₂ (black circles).

### DETAILED DESCRIPTION

Highly stable ionic liquids comprise an organic cation and an anion. The cations are nitrogen-containing five or six member ring heterocyclic compounds. For 6-member rings, pyrazinium and its derivatives are disclosed herein to be highly (‘super’) stable. For 5-member rings, pyrazolium and 1,2,4-triazolium and their derivatives are disclosed herein to be highly (‘super’) stable.

Stability as stated herein, refers to the ability of the reaction product (the anion-cation complex) to stay as a complex and not breakdown and release free anions and cations. As disclosed and shown herein, the release of NH₄⁺ ions is the benchmark for stability. That is, a cation such as pyrazole when incubated with an anion such as sulfuric acid, produces pyrazolium bisulfate, and because pyrazolium bisulfate is “stable”, the reaction product does not contain NH₄⁺ cations released from the amine group of the cation, as a breakdown product (breakdown species) in the reactant product solution.

The term ‘super’ as used herein, further qualifies the stability of the ionic liquids disclosed and claimed herein. Ionic liquids in general are known to be stable, however, the ionic liquids of the present disclosure (pyrazinium, pyrazolium and 1,2,4-triazolium and their derivatives) are ‘super’ stable because their stability is maintained under a combination of harsh conditions. That is, under high temperature, low pH and in the presence of a powerful transition metal catalyst, they remain as a catalyst and are not oxidized to reaction products-they are ‘super’ stable.

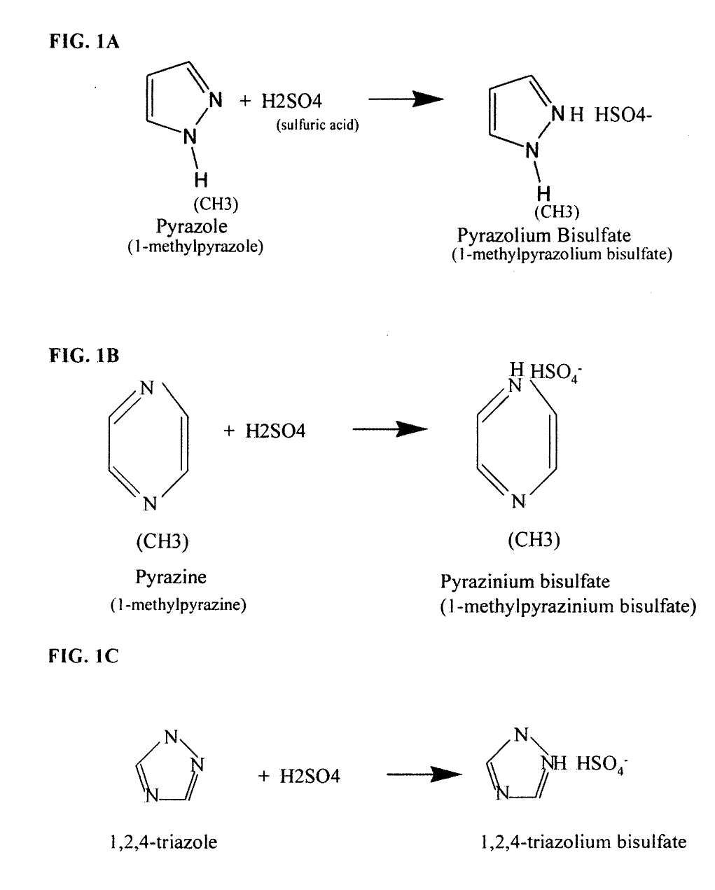

The derivatives of pyrazinium, pyrazolium and 1,2,4-triazolium of the present disclosure include, but are not limited to substituted carbon, hydrogen or other hetero-atoms at the ring carbon or ring nitrogen. The hetero-atoms include but are not limited to halides, oxygen, nitrogen, sulfur, phosphorous, silicane and metals. For dangling hydrocarbon alkyl groups attached to the ring, no group longer than one methyl (—CH₃) can be present if ‘super’ stability is to be maintained. An example of a methyl derivative is shown in FIG. 1A showing pyrazole and its methyl derivative 1-methylpyrazole in parentheses. The anions of the super stable ionic liquids include but are not limited to bisulfates, sulfates, nitrates, nitrites, triflates, acetates, azides, phosphates, and others, as would be well known to a person skilled in the art.

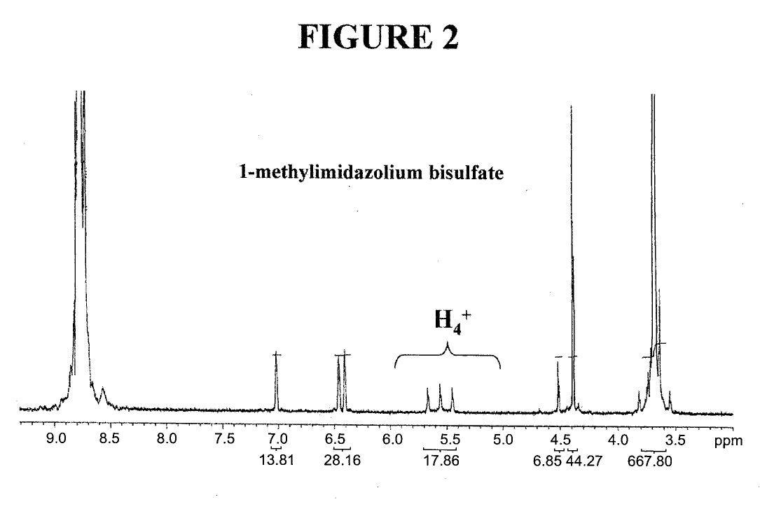

At high temperature (200° C.), in the presence of concentrated acid (94% H₂SO₄) and a powerful transition metal catalyst (PtCl₂) the commonly used and commercially available ionic liquid 1-methylimidizolium bisulfate is not stable. This is shown by ¹H-NMR (proton-nuclear magnetic resonance) with the presence of triple peaks between 5.4 and 5.7 ppm representing NH₄⁺ released from the amine group of the 1-methylimidizolium (FIG. 2).

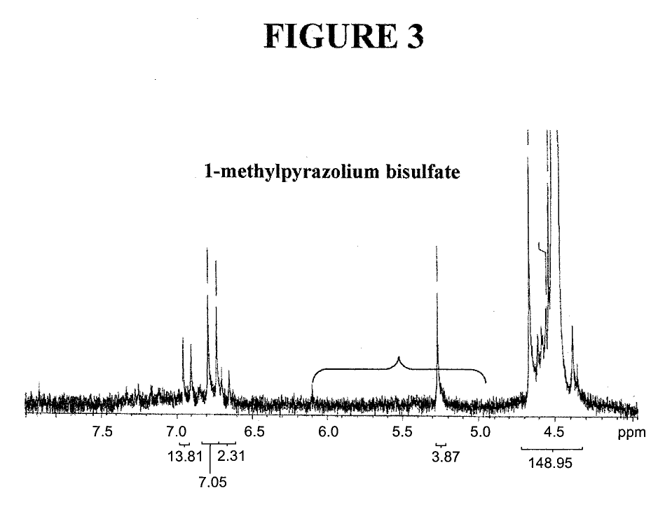

The bracket spanning from 5.0 to 6.0 in FIG. 2 is found in FIGS. 3-5. Since the scale of each NMR varies, the bracket size varies accordingly to show this 5.0 to 6.0 range in each figure.

Under the same or similar extreme conditions as for the 1-methylimidizolium bisulfate, the amine groups of the stable ionic liquids of the present invention are stable, as described herein. General methods for NMR analysis can be found in U.S. patent application Ser. No. 11/228,788, which is incorporated herein by reference in its entirety.

Stable Ionic Liquids Based on Pyrazole

The reaction of 1-methylpyrazole to 1-methylpyrazolium bisulfate is shown in FIG. 1A. This derivative of pyrazolium bisulfate is a highly stable ionic liquid because the structure does not release NH₄⁺ ions under harsh conditions. 1-methylpyrazolium bisulfate was dissolved with a platinum catalyst (reacted as PtCl₂) into concentrated sulfuric acid (102% H₂SO₄) and then heated to 200 degrees Celsius for 2.5 hours. Under these conditions, the 1-methylpyrazolium bisulfate maintained its structure, and the amine group was not oxidized to form NH₄⁺. As shown in FIG. 3, the NMR spectrum of 1-methylpyrazolium bisulfate does not contain the NH₄⁺ triple peaks between 5.4 and 5.7 ppm.

Stable Ionic Liquids Based on Pyrazine

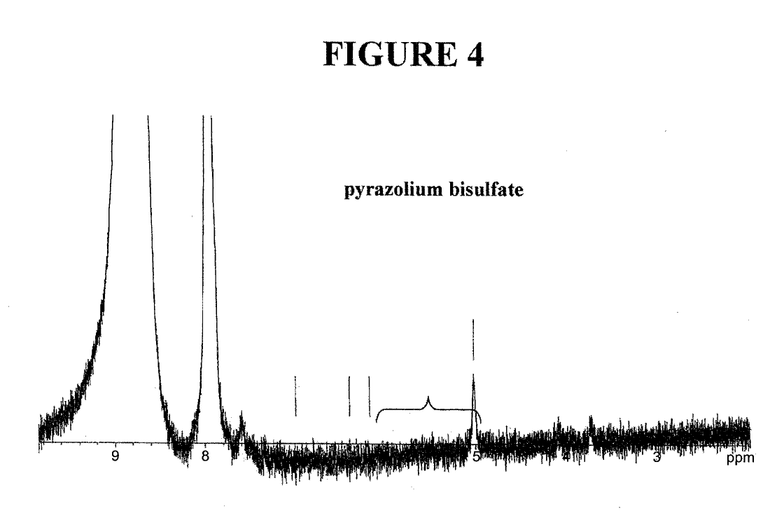

The reaction of pyrazine to pyrazinium bisulfate is shown in FIG. 1B. Pyrazinium bisulfate is a highly stable ionic liquid because it does not release NH₄⁺ ions under harsh conditions. Pyrazinium bisulfate was dissolved with PtCl₂ into concentrated sulfuric acid (97% H₂SO₄) and then heated to 200 degrees Celsius for 5.0 hours. Under these conditions, the pyrazinium bisulfate maintained its structure and the amine group was not oxidized to form NH₄⁺. As shown in FIG. 4, the NMR spectrum of pyrazinium bisulfate does not contain the NH₄⁺ triple peaks between 5.4 and 5.7 ppm.

Stable Ionic Liquids Based on 1,2,4-triazole

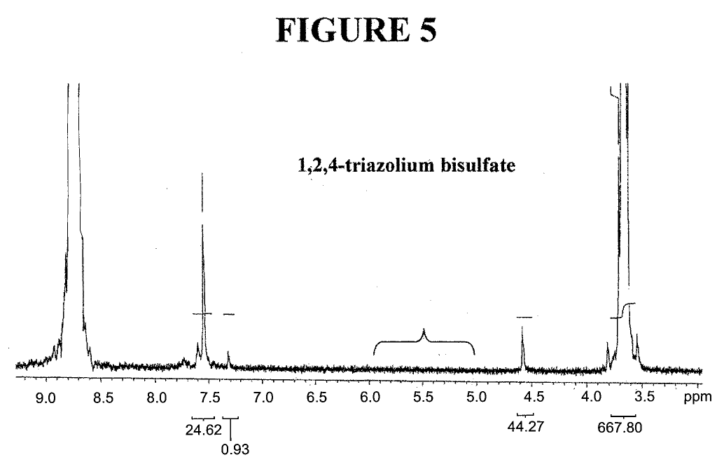

The reaction of 1,2,4-triazole to 1,2,4-triazolium bisulfate is shown in FIG. 1C. 1,2,4-triazolium bisulfate is a highly stable ionic liquid because it does not release NH₄⁺ ions under harsh conditions. 1,2,4-triazole was dissolved with PtCl₂ into concentrated sulfuric acid (102% H₂SO₄) and then heated to 200 degrees Celsius for 2.5 hours. Under these conditions, the 1,2,4-triazolium bisulfate maintained its structure, and the amine group was not oxidized to form NH₄⁺. As shown in FIG. 5, the NMR spectrum of 1,2,4-triazolium bisulfate does not contain the NH₄⁺ triple peaks between 5.4 and 5.7 ppm.

### EXAMPLES

For the reactions presented herein, 0.08 mol of the pyrazole, pyrazine or 1,2,4-triazole (or a derivative thereof) at room temperature was added to concentrated sulfuric acid (4.5 ml, 0.04 mol) in a small beaker with a magnetic stirrer and reacted at 200 degrees Celsius. After 2-24 hours, anhydrate ether (40 ml) was added and precipitated a light yellow oily phase. The liquid was removed with filtration and the precipitate was washed with ether. The precipitate was dissolved in 5 ml of methanol and then treated with ether to precipitate the IL-bisulfate compound which was then vacuum-dried.

In alternative methods, the reaction could take place under pressure, in both a low or high pressure reactor. Reaction temperatures for analyzing the stability of the ionic liquids of the present invention can range from 180-350 degrees Celsius.

In alternative methods, the concentrated acid can vary. Acids for analyzing the stability of the ionic liquids of the present invention include, but are not limited to sulfuric acid, nitric acid, phosphoric acid, hydrochloric acid, trifluoroacetic acid, triflic acid (CF₃SO₃H), oleum, selenic acid and water. The concentration of these acids can range up to 110%. Of course an ionic liquid of the present invention is also stable in dilute acid. For stability assays shown herein, concentrated acids were used, however, it is well known that dilute acids are preferred in many chemical reactions.

In alternative methods, the transition metal catalyst could be one of several known. Any derivative of Hg(II), Pt(II), Au(III) or PD(II) can be used. (Periana R. et al., Science, 1993, 259, 340-343; Periana R. et al., Science, 1998, 280, 560-564; Jones C. J. et al., Chem. Int. Ed, 2004, 43, 4626; Periana, R. et al., Science, 2003, 301, 814-817).

Applications

A ‘super’ stable ionic liquid like the ionic liquids described herein based on pyrazole, pyrazine, 1,2,4-triazole and their derivatives, can be used to improve a variety of applications. Examples are provided herein.

Chemical Processes—e.g. Methane to Methanol.

Methane oxidation to methanol is a reaction that is continually being improved upon. This reaction in concentrated H2SO4 using the “Periana” Pt (bpymPtCl2) catalyst as been previously described (Periana R. et al., Science, 1998, 280, 560-564). This methane oxidation reaction to methanol in concentrated H2SO4 using the “Periana” catalyst can be written as CH₄+2H₂SO₄→CH₃OSO₃H+2H₂O+SO₂ in which water is generated in situ during the reaction. The reactivity of (bpym)PtCl2 was found to be extremely sensitive to even small amounts of water. Quantum chemistry computations suggest that the water complex [(Hbpym)PtCl(H₂O)]²⁺ is about 30 kJ mol-1 more thermodynamically stable than the starting active Pt complex [(Hbpym)PtCl(H₂O₄)]²⁺, which is the so-called ground state effect for C—H activation (Periana R. et al., J. Mol. Catal. A: Chem., 2004, 220, 7; Kua, J., et al., Organometallics, 2002, 21, 51).

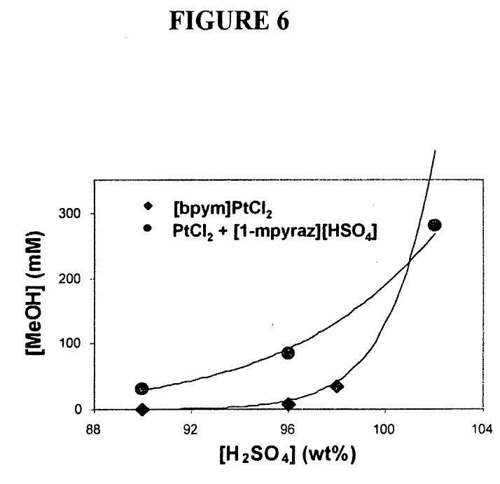

As shown in FIG. 6, the catalytic activity dropped sharply when the sulfuric acid was diluted from the oleum to below 100% (Cheng et al., Chem. Comm. 2006, 4617-4619). This decrease in activity and yield leads to uneconomical catalysis rates and high separation costs for the methanol. As a comparison, the Pt/1-methylpyrazinium/H₂SO₄ system at lower H₂SO₄ concentration (90 to 100%) exhibited higher methanol yields than the “Periana” catalyst. FIG. 6 shows the yield of methanol from methane as a function of H₂SO₄, at 200 degrees Celsius for 2.5 hours with 0.05 mmol Pt species 0.3 mmol IL at 500 psi methane. The reaction volume was 1 milliliter (mL) in a 69 mL reactor. The concentration of H₂SO₄ started at 102% and was diluted to 90%. This reaction in the presence of the “Periana” catalyst (bpym)PtCl₂ is shown as black diamonds and the 1-methylpyrazinium bisulfate (1-mpyraz)(HSO₄) is shown as black circles. This experiment shows that under more water tolerant conditions, more methanol can be produced. Given the ILs of the present disclosure are stable under such extreme conditions, they can be used in a variety of chemical processes.

Waste Treatment

The treatment of waste such as nuclear waste is difficult because a compound that has the potential to neutralize the waste usually can only do so at high temperatures or under acidic and oxidizing conditions, wherein the stability of many solvents (less stable ionic liquids) is compromised, or volatile. And, if temperatures are lowered, or pH is raised, then the solubility of the waste (e.g. uranium oxide) is compromised. ‘Super’ stable ionic liquids as those described in the present disclosure offer a less volatile solvent for the neutralization of waste.

Lithography

Components for UV lithography are such that any degradation in reflectivity of multilayer optics, such as would be caused by the formation of a carbon film, is unacceptable. Present methods for removing carbon by oxidation are difficult to apply in the environment of an EUV lithography system and are often harmful to optical components. Inherent with the stability of the stable ionic liquids of the present invention is that they are more inert and are therefore, less harmful. The stable ionic liquids of the present disclosure could be useful in preventing degradation of the optical components.

Hydrometallurgy

The extraction of metals, including rare metals from the earth can involve dissolving a metal oxide in acid in order to obtain the metal alone. Extreme conditions are often necessary. Ionic liquids which are soluble at room temperature offer a safer and environmentally cleaner approach for the extraction of metals. At present, a commercially available ionic liquid based on bis 2,4,4-trimethylpentylphosphinic acid is used to extract cobalt from cobalt/nickel solutions (WO 2003020843; U.S. Pat. No. 4,353,883). ‘Super’ stable ionic liquids of the present disclosure would provide a catalyst that could withstand more extreme extraction conditions.

Extension of Oxidation Potential

The ability to extend an oxidation potential in a battery or fuel cell is highly desirable. Ionic liquids have been used to extend the oxidation potential as battery electrolytes (Fannin, A. A et al., J. Phys. Chem. 1984, 88, 2610-2614; Fannin, A. A et al., J Phys. Chem. 1984, 88, 2614-2621). More stable ionic liquids could improve the extension of the oxidation potential.

Industrial Lubricants

Room temperature ionic liquids have been shown to be versatile industrial lubricants. These ILs show excellent friction reduction, anti-wear performance and high load-carrying capacity. Several have been used specifically for the contact of steel/steel, steel/aluminum, steel/copper, steel SiO2, Si3N4/SiO2, steel/Si(100), steel/sialon ceramics and Si3N4/sialon ceramics versatile lubricants for the contact of steel/steel, steel/aluminium, steel/copper, steel/SiO2, Si3N4/SiO2, steel/Si(100), steel/sialon ceramics and Si3N4/sialon ceramics (Ye, C et al. Chem. Commun. 2001, 21, 2244-2245).

While illustrative embodiments have been shown and described in the above description, numerous variations and alternative embodiments will occur to those skilled in the art. Such variations and alternative embodiments are contemplated, and can be made without departing from the scope of the invention as defined in the appended claims.

1. A stable ionic liquid complex comprising a cation and an anion; wherein the cation is selected from the group consisting of pyrazolium, pyrazinium and 1,2,4,-triazolium and derivatives of each said cation, and the anion is derived from an acid.

2. The stable ionic liquid complex of claim 1, wherein the derivatives are selected from the group consisting of substituted carbon, hydrogen, and hetero-atoms at the ring carbon or ring nitrogen of the cation; and wherein the hetero-atoms are selected from the group consisting of halides, oxygen, nitrogen, sulfur, phosphorous, silicane and metals.

3. The stable ionic liquid complex of claim 1, wherein the stable ionic liquid complex is stable up to 350 degrees Celsius.

4. The stable ionic liquid complex of claim 1, wherein the stable ionic liquid complex is stable in an acid concentration up to 110%.

5. The stable ionic liquid complex of claim 1, wherein the stable ionic liquid complex is stable in the presence of a transition metal catalyst.

6. The stable ionic liquid complex of claim 5, wherein the transition metal catalyst is selected from the group consisting of Hg(II), Pt(II), Au(III) and Pd(II).

7. The stable ionic liquid complex of claim 5, wherein the transition metal catalyst is Pt.

8. The stable ionic liquid complex of claim 1, wherein the acid is selected from the group consisting of sulfuric acid, nitric acid, phosphoric acid, hydrochloric acid, trifluoroacetic acid, triflic acid, oleum, selenic acid and water.

9. The stable ionic liquid complex of claim 1, wherein the stable ionic liquid complex is produced from a reaction; wherein said reaction produces a reaction product that lacks ammonium cations (NH₄⁺) at 200 degrees Celsius, in sulfuric acid in the presence of a Pt (II) transition metal catalyst.

10. The stable ionic liquid complex of claim 9, wherein the sulfuric acid has a concentration range of 90-110%.

11. The stable ionic liquid complex of claim 1, wherein said stable ionic liquid complex is provided in a process selected from the group consisting of a converting methane to methanol; neutralizing nuclear waste treatment; preventing degradation of optical components; metal extraction; extending oxidation potential of a battery, and industrial lubrication.

12. A method for converting methane to methanol comprising reacting methane in the presence of the stable ionic liquid complex of claim 1.

13. A method for treating nuclear waste comprising neutralizing the nuclear waste in the stable ionic liquid complex of claim 1.

14. A method for preventing degradation of optical components comprising providing the stable ionic liquid complex of claim 1 to the optical components.

15. A method of metal extraction comprising dissolving the metal contaminants in the stable ionic liquid complex of claim 1.

16. A method for extending oxidation potential of a battery comprising providing the stable ionic liquid complex of claim 1.

17. A method for providing an industrial lubricant comprising providing the stable ionic liquid complex of claim 1 as a lubricant.

18. A method for determining stability of an ionic liquid complex comprising reacting a cation selected from the group consisting of pyrazole, pyrazine, and 1,2,4-triazole and derivatives of each said cation, with an anion derived from an acid;
obtaining a reaction product containing the ionic liquid complex, and analyzing the reaction product for species other than the ionic liquid complex.

19. The method of claim 18 further comprising reacting the cation and anion in the presence of a transition metal at a temperature range between 200 and 350 degrees Celsius.

20. The method of claim 18, wherein analyzing the reaction product is carried out using nuclear magnetic resonance (NMR).

21. The method of claim 18, wherein the acid is sulfuric acid and the species other than the ionic liquid complex is ammonium.
